(19) United States
(12) Patent Application Publication
(10) Pub. No.: US 2024/0292818 A1
(43) Pub. Date: Sep. 5, 2024

(54) SYSTEMS AND METHODS FOR PRODUCING PROGENY WITH SELECTIVE TRAITS

(71) Applicant: Brigham Young University, Provo, UT (US)

(72) Inventors: Cayden Christopher Bro, Provo, UT (US); Jonathon Hill, Provo, UT (US); Jeffery Barrow, Provo, UT (US); Michael Stark, Mapleton, UT (US)

(21) Appl. No.: 18/593,658

(22) Filed: Mar. 1, 2024

Related U.S. Application Data
(60) Provisional application No. 63/449,184, filed on Mar. 1, 2023.

(51) Int. Cl. A01K 67/00; A01H 1/04; C12N 9/22; C12N 15/11

(52) U.S. Cl. CPC A01K 67/00; A01H 1/045; C12N 9/22; C12N 15/111; C12N 2310/20

(57) ABSTRACT

Systems and methods for producing organisms that lack one or more undesired traits are disclosed. In some implementations, a method includes obtaining (or producing) a first parent organism of a donor family of breeding stock and obtaining (or producing) a second parent organism from a receptor family of breeding stock. Some implementations of the method include mating the first parent organism with the second parent organism to produce a progeny organism. In some cases, a combination of genetic material from the first parent organism and the second parent organism in the progeny organism prevents development of the undesired trait (or of an organism having the undesired trait). Other implementations are also described.

### RELATED APPLICATION

This application claims priority to U.S. Provisional Patent Application No. 63/449,184, filed Mar. 1, 2023, entitled “Reagents and Methods of BRO-LESS: a Cas9 Method to Direct Embryo Arrest”, which is incorporated by reference herein in its entirety.

### FIELD

The present disclosure relates generally to the field of selective breeding, and more particularly to systems and methods for selecting for specific traits in progeny through use of molecular mechanisms introduced via genetic modification of breeding stock organisms.

### BACKGROUND

Selective breeding programs traditionally seek to reproduce desirable traits in organisms while eliminating undesirable ones. This is typically done by selecting organisms having the desirable traits and allowing or causing them to reproduce with each other, while eliminating or disqualifying from mating organisms having the undesired traits. Often, organisms with undesired traits are culled, which can incur significant labor and costs, and which is seen by many as being cruel or inhumane. Some countries have even enacted legislation to prevent or restrict certain types of culling, creating a need for alternative solutions.

In many cases, traits that are desirable or necessary in breeding stock are undesirable in progeny. For example, it may be necessary for breeding stock plants to produce seeds, while it may also be desirable for progeny plants to be seedless (e.g., to increase the appeal of fruit consumption). As another example, both roosters and chickens are typically necessary to produce offspring chicks in egg and poultry industries, but it is often the case that only the female chicks are needed or desired. As a result, a common practice in the poultry industry is to cull rooster chicks (e.g., day-old male chicks) through incineration, grinding, drowning, or other culling processes. Similarly, cattle breeding for dairy farmers generally requires both bulls and cows, but since only cows can produce milk, male offspring are often culled. Thus, a significant investment of labor and resources is often dedicated to producing and eliminating nonprofitable organisms.

Thus, while techniques currently exist that are used to eliminate undesired traits in progeny, challenges still exist, including those listed above. Accordingly, it would be an improvement in the art to augment or even replace current techniques with other techniques.

### SUMMARY

Systems and methods for producing organisms that lack one or more undesired traits are disclosed. In some implementations, a method includes obtaining (or producing) a first parent organism of a donor family of breeding stock and obtaining (or producing) a second parent organism from a receptor family of breeding stock. Some implementations of the method include mating the first parent organism with the second parent organism to produce a progeny organism. In some cases, a combination of genetic material from the first parent organism and the second parent organism in the progeny organism (e.g., as naturally occurs during the process of sexual reproduction) prevents development of the undesired trait (or of an organism having the undesired trait). In some cases, this is due to one or more genetic differences between the donor family and the receptor family of breeding stock. In some cases, this is due to a non-native (e.g., not normally present in organisms of the type in question) genetic construct present in the genetic material.

In some implementations, at least one of the first parent organism and the second parent organism has one or more alleles that correspond to the undesired trait. In some cases, at least one of the first parent organism and the second parent organism has the undesired trait (but in some cases, neither parent organism has the undesired trait, even though at least one of the parents has the one or more alleles that correspond to the undesired trait).

According to some implementations, the combination of genetic material from the first parent organism and the second parent organism in the progeny organism prevents development of the undesired trait by arresting development of the progeny organism if the progeny organism has an allele that corresponds to the undesired trait. According to other implementations, if the progeny organism has the allele that corresponds to the undesired trait, the combination of genetic material from the first parent organism and the second parent organism in the progeny organism prevents development of the undesired trait by causing the allele that corresponds to the undesired trait in the progeny organism to be modified (e.g., by triggering modification of the allele using natural or artificial cellular modification mechanisms).

In some implementations, the first parent organism has a genetic construct. In some cases, the genetic construct includes a Cas9 coding region (which, in some cases, is configured to produce a Cas9 protein). In some cases, the genetic construct includes a guide RNA coding region (which, in some cases, is configured to produce guide RNA configured to associate with the Cas9 protein). In some iterations, the Cas9 coding region is linked to an early embryonic promoter (e.g., to ensure transcription of the Cas9 protein at an early stage of the progeny organism's development). In some cases, the genetic construct includes the early embryonic promoter. Additionally, some iterations of the genetic construct include a U6 promoter (which, in some cases, is linked to the guide RNA coding region).

With further respect to the guide RNA, some implementations of the guide RNA coding region are configured to result in production of guide RNA that is configured to interact (e.g., match or align) with a complementary genetic sequence. In some cases, the complementary genetic sequence is not present in DNA of the first parent organism, but (in some cases) it is present in DNA of the second parent organism.

In some implementations, the complementary genetic sequence includes a polymorphism of a gene. In some cases, the gene is present in the DNA of the first parent organism and in the DNA of the second parent organism (although in some cases, the gene is present in only one of the parent organisms). In some implementations, the polymorphism comprises a silent mutation in a nucleotide sequence of the gene (e.g., a mutation that does not, under normal conditions, affect the gene).

According to some implementations, the genetic construct is present in (e.g., inserted into) a genetic safe harbor of DNA of the first parent organism (e.g., a region of DNA that, if disrupted, does not adversely affect the organism). According to other implementations, the genetic construct is present in a locus of the gene. In some cases, this results in a neutralized (e.g., mutated, inactive, or otherwise disrupted) allele of the gene. According to still other implementations, the construct is located in genetic proximity (e.g., close together within a DNA sequence, rather than close physically, although the proximity can also be physical) to a target allele. In some cases, this is done such that activation of the construct is linked to inheritance and activation of the allele.

Although the undesired trait can be any trait or characteristic of an organism, in some cases the undesired trait includes a sex (e.g., male, female) of the progeny organism.

According to some embodiments, disclosed is a method of producing breeding stock configured to produce progeny that lack an undesired trait when a member of a donor family of breeding stock is mated with a member of a receptor family of breeding stock (e.g., in accordance with the foregoing). In some cases, the method includes one or more of the following: obtaining a first target organism; obtaining a first knock-in construct (which in some cases includes a Cas9 coding region configured to result in production of a Cas9 protein, and a guide RNA coding region configured to result in production of a guide RNA); introducing the first knock-in construct into a primordial germ cell of the first target organism (e.g., using a first CRISPR knock-in procedure); allowing the first target organism to mature; selectively breeding the first target organism to produce the donor family of breeding stock such that each member of the donor family of breeding stock comprises the first knock-in construct; obtaining a second target organism; obtaining a second knock-in construct comprising a nucleotide sequence that is complementary to the guide RNA; introducing the second knock-in construct into a primordial germ cell of the second target organism (e.g., using a second CRISPR knock-in procedure); allowing the second target organism to mature; and selectively breeding the second target organism to produce the receptor family of breeding stock such that each member of the receptor family of breeding stock comprises the second knock-in construct.

In some implementations, the first knock-in construct further comprises one or more of a first homology arm and a second homology arm. In some cases, one or each of the first homology arm and the second homology arm are complementary to a genomic safe harbor in a genome of the first target organism.

In some iterations, the Cas9 coding region of the first knock-in construct is linked to an early embryonic promoter. In some cases, this is done such that the donor family of breeding stock is configured to produce the Cas9 protein during an early stage of embryonic development.

In some iterations, the second CRISPR knock-in procedure results in a silent mutation in a target gene of the second target organism. In some cases, the silent mutation includes an insertion of the second knock-in construct.

According to some implementations of the systems and methods discussed herein, a bifurcated breeding stock for producing progeny that lack an undesired trait is disclosed. In some implementations, the bifurcated breeding stock includes one or more donor families and one or more receptor families. In some implementations, the donor family has a first construct, which in some cases includes: a Cas9 coding region configured to result in production of a Cas9 protein; and a guide RNA coding region configured to result in production of a guide RNA configured to associate with the Cas9 protein. In some implementations, the receptor family has a second construct, which in some cases includes a nucleotide sequence that is complementary to the guide RNA.

In some cases, breeding members of the donor family with other members of the donor family produces donor family offspring. Additionally, in some cases, breeding members of the receptor family with other members of the receptor family produces receptor family offspring. This notwithstanding, in some cases, breeding a member of the donor family with a member of the receptor family produces only progeny that lack the undesired trait in question (e.g., due to an interaction between the Cas9 protein and the nucleotide sequence that is complementary to the guide RNA in progeny that would otherwise have the undesired trait).

According to some implementations, the interaction between the Cas9 protein and the nucleotide sequence that is complementary to the guide RNA in progeny that would otherwise have the undesired trait results in arrested embryonic development of the progeny that would otherwise have the undesired trait. In some implementations, progeny that would not otherwise have the undesired trait are not affected by (or not subjected to at all, in some cases) the interaction between the Cas9 protein and the nucleotide sequence that is complementary to the guide RNA. Thus, in some cases, progeny without the trait develop normally, while progeny that would have the trait are prevented from developing at the embryonic stage.

In some implementations, the undesired trait is a sex of the progeny, and in some implementations, at least one of the first construct and the second construct is present within a genetic sequence of a sex chromosome (e.g., X, Y, Z, W, or other sex chromosomes depending on the organism in question).

### BRIEF DESCRIPTION OF THE DRAWINGS

The objects and features of the present disclosure will become more fully apparent from the following description and appended claims, taken in conjunction with the accompanying drawings. Understanding that these drawings depict only typical embodiments of the disclosed systems and methods and are, therefore, not to be considered limiting of its scope, the systems and methods will be described and explained with additional specificity and detail through the use of the accompanying drawings in which:

FIG. 1 shows a pedigree chart, in accordance with a representative embodiment of the disclosed systems and methods;

FIG. 2 shows a schematic diagram of a donor construct, in accordance with a representative embodiment;

FIG. 3 shows an illustrative comparison of a receptor construct with a wild-type genetic sequence, in accordance with a representative embodiment;

FIG. 4 shows a graphic depicting the basic functionality of a Cas9 protein, in accordance with a representative embodiment;

FIG. 5 shows a graphic illustrating the results of the possible combinations of donor genetic material with receptor genetic material, in accordance with a representative embodiment;

FIG. 6 shows a graphic illustrating genetic material of a donor breeding stock and of a receptor breeding stock, in accordance with another representative embodiment; and

FIG. 7 shows a graphic illustrating genetic material of a donor breeding stock and of a receptor breeding stock, in accordance with another representative embodiment.

### DETAILED DESCRIPTION

A description of embodiments will now be given with reference to the Figures. It is expected that the present systems and methods may take many other forms and shapes, hence the following disclosure is intended to be illustrative and not limiting, and the scope of the disclosure should be determined by reference to the appended claims.

The instant systems and methods relate generally to selective breeding, or breeding organisms to preserve or eliminate certain traits. Although they may be particularly useful in plant and animal industries (e.g., relating to food production, cosmetic production, medical compound production, or other plant or animal product production), the systems and methods disclosed herein can be used in connection with any organisms capable of performing sexual reproduction or other processes involving the combination of two separate sets of genetic material. For example, the systems and methods described herein can be used in connection with fungi, protists, bacteria, plants (e.g., fruit-and vegetable-yielding plants, flowering plants, trees, shrubs, and any other suitable types of plants), and animals (e.g., livestock, such as cows, goats, horses, pigs, and any other livestock; poultry, such as chickens, ducks, turkeys, peacocks, pheasants, and any other poultry; pets, such as dogs, cats, lizards, snakes, and any other pets; research animals, such as mice, rats, monkeys, and any other research animals; fish; zoo animals; and any other animals).

According to some embodiments, the instant systems and methods can be used to ensure that progeny (e.g., organisms produced as a result of breeding parent organisms) have or lack certain traits. For example, progeny organisms can be produced that lack a trait, even if one or both of the parent organisms have the trait or have alleles (variants of genes) that typically produce progeny with that trait. As another example, progeny organisms can be produced that have a trait, even if one or both of the parent organisms lack the trait or lack alleles that could naturally give rise to that trait.

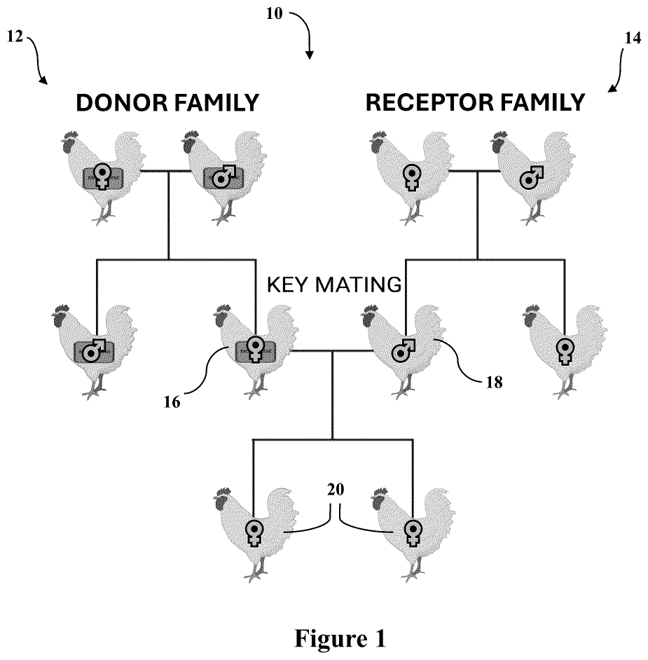

To provide a non-limiting example to lend some context to the foregoing and following disclosure, FIG. 1 shows a pedigree chart in which two separate families of breeding stock 10 (a donor family 12 and a receptor family 14) of organisms (chickens, in the case of FIG. 1) exist. Each family of chickens has a mixture of traits. In FIG. 1, the trait of interest is biological sex, with male chickens (roosters) represented by the ♂ symbol, and female chickens (hens) represented by the ♀ symbol. When members of the donor family reproduce with other members of the donor family, the resulting progeny have a normal mixture of the trait (i.e., both roosters and hens are produced). Similarly, when members of the receptor family reproduce with other members of the receptor family, the resulting progeny have a normal mixture of the trait (i.e., both roosters and hens are produced). However, when a first parent 16 (a member of the donor family) is mated with a second parent 18 (a member of the receptor family), the resulting progeny 20 all have a desired trait (in the case of FIG. 1, a female genotype, so the progeny are all hens) and lack an undesired trait (in the case of FIG. 1, a male genotype, so there are no roosters among the progeny).

It is worth noting that “desired traits” and “undesired traits” are not limited to beneficial and detrimental traits, respectively. Rather, these terms simply relate to the attributes desired or not desired in the specific progeny in the specific instance. For example, in many cases it is desirable for roosters to exist in the donor family and the receptor family (in order to allow those families to continue to reproduce and be self-sustainable), but it can also be desirable to produce progeny that lack males as a product of mating members of the two families (e.g., for purposes of egg production, poultry production, or any other suitable purpose). Accordingly, the desired traits obtained through use of the present systems and methods, as well as the undesired traits eliminated therewith, can include any trait of a user's selection. Non-limiting examples of such traits include the following: sex; size; height; weight; coloring; appearance; production capacity (e.g., of eggs, meat, milk, fruit, wool, fibers, proteins, fats, oils, carbohydrates, chemical compounds, vitamins, polymers, or any other products of organisms); growth rate; abnormalities (e.g., cancer growths, hormone imbalances, diseases, disabilities, or other abnormalities, which may be desirable to eliminate in many cases, or which may be desirable to reproduce for research or similar purposes); metabolic properties; strength; social habits; intellectual capacity; selective responses to stimuli; other social, physical, or mental traits or capabilities; and any other suitable traits.

At a high level, some embodiments of the systems and methods of this disclosure include a method of breeding organisms to produce progeny that lack an undesired trait (or that have a desired trait). In some embodiments, the method includes obtaining a first parent organism 16 from a donor family 12 of breeding stock 10 and obtaining a second parent organism 18 from a receptor family 14 of the breeding stock. As discussed in greater detail below, the method can include producing the breeding stock. Thus, “obtaining” the parent organisms can include producing the parent organisms, obtaining the parent organisms from previously formed breeding stock, or otherwise obtaining the parent organisms.

In some embodiments, the method includes mating the first parent organism 16 with the second parent organism 18 to produce a progeny organism 20, wherein a combination of genetic material (e.g., DNA) from the first parent organism and the second parent organism in the progeny organism prevents development of the undesired trait (or development of progeny that would otherwise have the undesired trait). In this regard, when a progeny organism is produced via sexual reproduction, the progeny organism typically receives approximately half of its genetic material from its first parent (e.g., its mother or father) and approximately half of its genetic material from its second parent (e.g., if the first parent is the mother, then the second parent is the father, and vice versa). The organism then develops using its unique set of combined genetic material as developmental instructions. The presently disclosed method generally takes advantage of this combination of DNA (forming a new set of genetic material) to implement molecular mechanisms (as discussed in more detail below) that ensure the progeny has the desired trait or lacks the undesired trait.

Referring generally to FIGS. 1-3, and regarding the particular molecular mechanisms involved, in some embodiments, one or more members of the breeding stock 10 have one or more genetic constructs 22 included in their genomes. The term “genetic construct” is generally used herein to refer to a sequence of DNA that is included at a particular locus within an organism's genome, and which is not normally present at that locus in wild-type (WT) organisms of the organism's species. The genetic construct can include any suitable construct for ensuring that desired traits are expressed or that undesired traits are not expressed in the progeny 20 of a first parent 16 (selected from a donor family 12) and a second parent 18 (selected from a receptor family 14). In some cases, the donor family has a first genetic construct. In some cases, the receptor family has a second genetic construct. In some embodiments, it is the inheritance of both the first genetic construct and the second genetic construct in at least some progeny resulting from mating members of both families that leads to the full group of progeny having or lacking (as applicable) the target trait.

In some embodiments, the first genetic construct comprises a Cas9 construct 24. In some embodiments, the second genetic construct comprises a complementary sequence construct 26. It can also be the other way around: in some embodiments, the first genetic construct comprises a complementary sequence construct and the second genetic construct comprises a Cas9 construct.

The Cas9 construct 24 can be any suitable construct having a Cas9 coding region 28, meaning any suitable sequence of DNA configured to code for and produce (in connection with the organism's native cellular machinery) a Cas9 protein. For example, the construct 24 can include a sequence of DNA, which, after transcription (creation of an RNA sequence using the DNA template), excision of introns (cutting out unneeded portions, if any), translation (building of a polypeptide chain based on the RNA sequence), and folding (folding the polypeptide chain and combining it with other amino acid sequences, if necessary), forms a Cas9 protein.

In some embodiments, the Cas9 construct 24 includes a guide RNA coding region 30, or any suitable sequence of DNA configured to code for a guide RNA. In particular, the guide RNA coding region codes, in at least some embodiments, for a guide RNA that is configured to associate with (e.g., bind to) the Cas9 protein and to direct the Cas9 protein to a particular complementary DNA sequence.

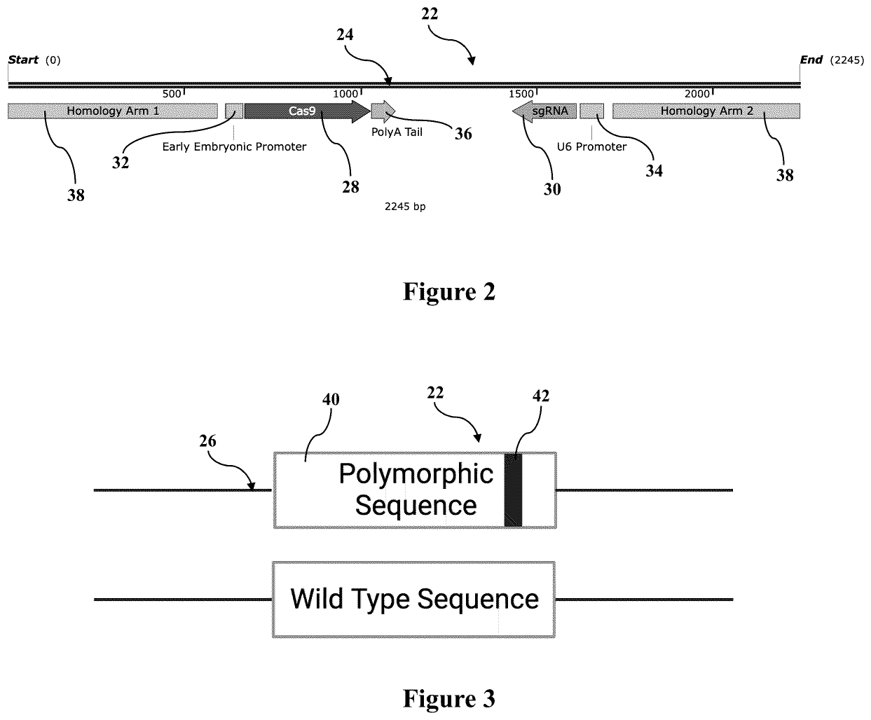

In some embodiments, one or more of the Cas9 coding region 28 and the guide RNA coding region 30 is linked to one or more promoters (e.g., the promoter is included within the Cas9 construct 24 in genetic proximity to the region in question, such that activation of the promoter triggers transcription of the region). In this regard, a promoter is, in accordance with some embodiments, a region of DNA that triggers transcription at certain times or under certain conditions (for example, a certain promotor may trigger production of a specific protein at a certain time during an organism's development). The promoter can be any suitable promoter, such as: one or more core promoters; proximal promoters; distal promoters; prokaryotic promoters; eukaryotic promoters; promoters that can be selectively activated or deactivated by simulating certain conditions (e.g., addition or removal of suitable biological compounds); developmental promoters; organism-specific promoters; and any other constitutive, inducible, specific, or other promoters that can be used to trigger production of Cas9, guide RNA, or other components. As non-limiting examples, promoters that might be used include: U6, U3, CMV, EF1a, CBh, Tet-ON, CAG, PGK, TRE, UAS, T7, Sp6, lac, araBad, trp, Ptac, nos, CaMV, Ubi-1, rbcS, or any other suitable promoters. By way of non-limiting illustration, FIG. 2 shows a Cas9 construct 24 in which a Cas9 coding region 28 is linked to an early embryonic promoter 32 (to trigger production of Cas9 early on in embryonic development), and a guide RNA coding region is linked to a U6 promoter 34 (which is a constitutively active promoter—one that is active under all or almost all cellular conditions—to ensure that an ample amount of guide RNA is available as needed).

In some embodiments, the Cas9 construct 24 includes one or more additional components. For example, some embodiments include one or more poly-a tail regions 36 (which can help to stabilize RNA molecules). Some embodiments include one or more homology arms 38 (which can help ensure that the construct is inserted into a target portion of the organism's genome and not another region, as discussed in more detail later). Some embodiments include one or more portions that are complementary to a protospacer adjacent motif (PAM) configured to be recognized by the Cas9 protein.

In some embodiments, the complementary sequence construct 26 includes any genetic construct where at least a portion of the genetic construct includes a complementary genetic sequence 40 that is complementary to a guide RNA (such that the guide RNA associates with the complementary genetic sequence and, if the guide RNA is associated with Cas9, directs the Cas9 to the region of DNA with the complementary genetic sequence).

Some embodiments of the complementary sequence construct 26 contain one or more polymorphisms 42 (differences from a WT version of the gene) in the complementary genetic sequence, such that the complementary genetic sequence is not found in WT organisms. In some embodiments, the polymorphism comprises one or more silent mutations (e.g., an addition to, deletion from, or other change in the genetic sequence that does not affect the gene in question). Thus, in some embodiments, the organism with the complementary genetic sequence is unaffected by the presence of the complementary genetic sequence unless Cas9 (with a guide RNA that is complementary to the complementary genetic sequence) is also present.

Some embodiments of the complementary sequence construct 26 include additional components, such as any of the additional components that might be present in the Cas9 construct (e.g., homology arms). In some embodiments, the complementary sequence construct includes one or more PAM sites for recruitment of Cas9. In some cases, the PAM site includes one or more polymorphisms, which in some cases are configured to generate resistance to Cas9.

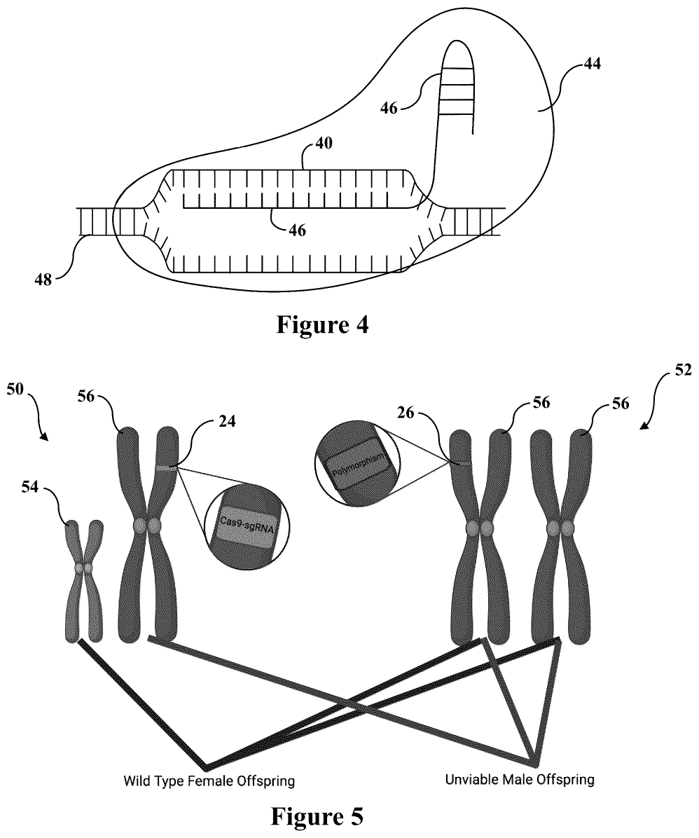

FIG. 4 provides some additional context for how the molecular mechanisms disclosed herein function. In this regard, FIG. 4 shows a Cas9 protein 44 (such as can be produced based on a Cas9 coding region 28 of a genetic construct 22 inherited from a first parent 16). Associated with the Cas9 protein is a guide RNA 46 (such as can be produced based on the guide RNA coding region 30 of the genetic construct 22 of a first parent). The guide RNA 46 is aligned with a complementary genetic sequence 40 located on one strand of a double stranded DNA molecule 48 (such as a complementary genetic sequence 40 of the genetic construct 22 inherited from a second parent 18). Thus, where the Cas9 protein 44, the guide RNA 46, and the complementary genetic sequence 40 are all present together (e.g., where each of these components is inherited from one parent or the other), the guide RNA 46 guides the Cas9 protein 44 to the strand of the DNA molecule 48 at the complementary genetic sequence 40. The Cas9 protein 44 then cuts (or induces a double stranded break, or otherwise breaks, mutates, or modifies) the DNA molecule 48. Accordingly, when the complementary genetic sequence 40 is placed in a certain gene, a certain allele of a gene, or any other suitable genetic sequence, the modifications performed by the Cas9 protein 44 can break, mutate, knock-out, modify, or otherwise neutralize the gene, allele, or genetic sequence.

The results of the foregoing process, according to some embodiments, are further illustrated in FIG. 5. FIG. 5 shows a first set of chromosomes 50 (e.g., chromosomes of a first parent 16) and a second set of chromosomes 52. Although the chromosomes can be any chromosomes of any organism, the chromosomes illustrated in FIG. 5 include avian sex chromosomes. In this regard, avian sex chromosomes typically include a W chromosome 54 and a Z chromosome 56. When a bird has one W chromosome and one Z chromosome, the bird is a female, whereas birds that have two Z chromosomes are male. Thus, the first set of chromosomes 50 are the chromosomes of a female, and the second set of chromosomes 52 are those of a male. When the two parents mate with each other, each child among the progeny receives one chromosome from the mother and one from the father. In the case of FIG. 5, the W chromosome is a WT chromosome, while the Z chromosomes are modified: the Z chromosome of the female parent includes the Cas9 construct 24, and at least one of the Z chromosomes of the male parent (and in some cases, both Z chromosomes) includes a complementary sequence construct 26. Thus, where offspring receive a W chromosome from the female parent and either Z chromosome from the male parent, the offspring have no Cas9 construct 24, and develop as normal females. Similarly, where offspring receive the Z chromosome from the female parent and a Z chromosome that does not have the complementary genetic sequence from the male parent, the offspring have the Cas9 construct 24 but the resultant Cas9 is not directed to any particular location on the DNA (because there is no complementary genetic sequence 40), and the resultant male progeny develop normally. However, where offspring receive the Z chromosome from the female parent and a Z chromosome having the complementary genetic sequence 40 from the male parent, the Cas9 in the offspring is localized to the complementary genetic sequence 40, and the target gene is deactivated or modified. In the case of FIG. 5, the resultant modification leads to unviable embryos, so the development of males receiving the combination is arrested, and the male progeny is unviable. Accordingly, this process can be used to reduce the number of male chicks (if the complementary genetic sequence 40 is present on only one of the male parent's Z chromosomes) or eliminate male chicks among the progeny (if both of the male parent's Z chromosomes contain the complementary genetic sequence 40).

It is worth reiterating that this process is not limited to avian chromosomes. Indeed, a similar method can be employed in mammalian chromosomes, plant chromosomes, or any other suitable chromosomes, which can vary broadly from organism to organism. For example, mammalian sex chromosomes typically include X chromosomes and Y chromosomes, with mammals having two X chromosomes being females and mammals having an X chromosome and a Y chromosome being males. Thus, dairy cows could be produced, for example, without the need to cull bulls from the all-female offspring, by including the Cas9 construct 24 on a male parent's Y chromosome and complementary sequence constructs 26 on the female parent's X chromosomes (or vice versa). As another example, if a breeder desired to produce a plant with all-white flowers, complementary genetic sequences 26 could be coupled to alleles resulting in alternative coloration (e.g., alleles that result in yellow flowers); thus Cas9 (received from one parent) would localize to the undesired yellow-flower alleles (thanks to the complementary genetic sequence 40 received from the other parent) and disable the undesired alleles (either modifying the flowers that would have been yellow to have only white coloration, or making plants with yellow flowers unviable, so that only white-flowered plants survive to maturity). Thus, in some embodiments, the combination of genetic material from the first parent organism and the second parent organism in the progeny organism prevents development of the undesired trait by arresting development the progeny organism if the progeny organism has an allele that corresponds to the undesired trait. Yet, in some embodiments, if the progeny organism has an allele that corresponds to the undesired trait, the combination of genetic material from the first parent organism and the second parent organism in the progeny organism prevents development of the undesired trait by causing the allele that corresponds to the undesired trait in the progeny organism to be modified. Again, this method can broadly apply to any suitable alleles of any suitable genes of any suitable chromosomes (not limited to sex chromosomes) of any suitable organisms, thus being useful for targeting an extremely broad array of traits. It is also worth noting that the allele can directly or indirectly correspond to the target trait (e.g., the allele can code for a protein that directly results in the desired trait, or the allele can code for a protein that influences the desired trait in any manner, such as through epistasis).

Although in some embodiments the complementary sequence construct 26 is included within or near a gene that is desired to be neutralized (e.g., so that the Cas9 protein will cut or otherwise modify the gene or a sequence near the gene that causes the gene to be neutralized), in some embodiments the complementary sequence construct 26 is included elsewhere within the genetic code (e.g., within a promoter region, within another gene that affects expression of the target gene, or in another location capable of bringing about or furthering a desired effect). Additionally, any number of complementary genetic sequences 40 can be included (thereby allowing for targeting multiple areas of DNA within a single organism). Where multiple complementary genetic sequences 40 are included, the sequences can be identical (e.g., so that a single guide RNA can direct Cas9 to all of them) or they can differ (so that multiple guide RNAs are required to direct Cas9 to the various genetic loci). Correspondingly, the Cas9 construct 24 can include a single guide RNA coding region or multiple of them.

Likewise, the Cas9 construct 24 can be included at any suitable locus in an organism's genetic code. Indeed, different positioning of the construct 24 can lead to different effects. In this regard, some embodiments include the Cas9 construct 24 in a genetic safe harbor (GSH), meaning an area of genetic code that, if disrupted, does not adversely affect the organism in question (e.g., a non-coding region of DNA). This can help to ensure that the newly inserted genetic elements function predictably and do not cause alterations of the host genome that pose an unintentional risk to the host cell or organism. In some embodiments, this leads to a dominant lethal mutation, as shown in FIG. 5.

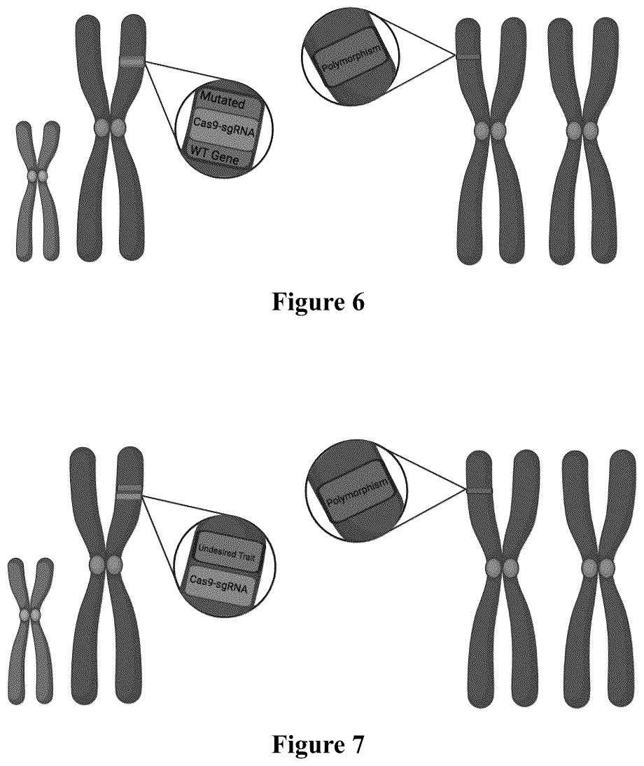

In other embodiments, as shown in FIG. 6, the Cas9 construct 24 can be inserted into the middle of a target gene, thereby mutating it. In some embodiments, this leads to a recessive system of inheritance: the gene on the chromosome of one parent is mutated and neutralized through insertion of the Cas9 construct 24, and the guide RNA 30 will target the complementary genetic sequence 40 on the other parent, which, when mutated, ensures that the offspring inherits zero functional copies of the target gene (and, if the gene is necessary for the organism to develop, the resulting organism will fail to develop).

In still other embodiments, as shown in FIG. 7, instead of (or in addition to) inserting the Cas9 construct 24 in a GSH or inside of a gene locus, the Cas9 construct 24 can be placed in a noncoding region of the genome close to the target gene, such that the construct and the gene are linked (thereby ensuring that inheritance of the selected trait will also bring inheritance of the Cas9 complex to arrest the embryo or modify the gene). This embodiment may be of particular interest when trying to remove certain traits from a community of organisms.

According to some embodiments of the present systems and methods, the foregoing method includes (or another method is provided for) producing breeding stock 10 (such as the breeding stock having any of the characteristics discussed herein, namely breeding stock configured to produce progeny that lack an undesired trait or have a desired trait when a member of a donor family of breeding stock is mated with a member of a receptor family of breeding stock).

In some embodiments, the method includes obtaining one or more first target organisms and one or more second target organisms (which can be any suitable organisms of the user's choosing). In some embodiments, the organisms comprise WT organisms. In some embodiments, the organisms comprise modified organisms (for example, if the organisms already have a genetic construct inserted into their genetic code, and a second genetic construct is desired to be inserted).

In some embodiments, the method includes obtaining a first knock-in construct, which in some cases is a Cas9 construct (including any of the features of such a construct, as discussed above). In some embodiments, obtaining the first knock-in construct includes designing the construct or any portion thereof (e.g., designing the guide RNA coding portion to correspond with a particular corresponding genetic sequence). In some embodiments, obtaining the first knock-in construct includes forming the first knock-in construct (through any suitable method of creating a genetic construct, such as gene synthesis, plasmid synthesis, gene splicing, and any other method).

In some embodiments, the method includes introducing the first knock-in construct into a primordial germ cell of the first target organism. Although this can be done in any suitable manner, in some embodiments, this is done using a CRISPR knock-in procedure. For example, in some embodiments this includes injecting (or otherwise introducing into the primordial germ cell) Cas9 protein with guide RNA configured to direct the Cas9 protein to the desired site of insertion to create a double-strand break in the DNA at a target locus (such as a GSH, gene locus, or non-coding region of DNA), as well as injecting (or otherwise introducing) the knock-in construct, ensuring that homology arms of the knock-in construct align with the ends of the broken portion of DNA, such that cellular repair mechanisms insert the construct between the ends of the double-strand break.

In some embodiments, the method includes obtaining a second knock-in construct comprising a nucleotide sequence that is complementary to the guide RNA, and introducing the second knock-in construct into a primordial germ cell of the second target organism (using any suitable method, such as another CRISPR knock-in procedure). In some embodiments, use of the CRISPR knock-in procedure is configured to produce a silent mutation that includes an insertion of the second knock-in construct.

In some embodiments, the method includes allowing the first target organism to mature, then selectively breeding the first target organism to produce the donor family of breeding stock (such that each member of the donor family of breeding stock has the first knock-in construct). For example, this selective breeding can include genetically testing the progeny of the first target organism (e.g., via a polymerase chain reaction (PCR) method or any other suitable method) to determine whether such progeny have the genetic construct, and breeding only the progeny that have the construct with each other until a breeding stock that is homozygous (or heterozygous, if desired) for the construct is produced. Similarly, some embodiments of the method include allowing the second target organism to mature and selectively breeding the second target organism to produce the receptor family of breeding stock (such that each member of the receptor family of breeding stock comprises the second knock-in construct). In some embodiments, the two families of breeding stock are kept separate from each other to prevent interbreeding until such interbreeding is desired. In some embodiments, use of the method allows for the donor family of breeding stock to breed with each other normally to produce new members of the donor family, and for members of the receptor family of breeding stock to likewise breed with each other normally to produce new members of the receptor family.

According to some embodiments of the systems and methods disclosed herein, the breeding stock 10 itself is provided. In this regard, some embodiments of the breeding stock include a bifurcated breeding stock (i.e., divided into a donor family and a receptor family), which can have any of the features discussed in connection with the methods disclosed herein.

The described systems and methods can be modified in any suitable manner. For instance, although this disclosure often discusses use of the systems and methods herein in connection with a single desired or undesired trait, it is important to note that the systems can be used to ensure that the progeny has or lacks multiple traits simultaneously (or sequentially). For example, in some cases a genetic construct can be used which targets multiple genes. In some cases, targeting a single gene can influence multiple traits. In some embodiments, multiple constructs are used (e.g., multiple Cas9 constructs, multiple differing guide RNA coding regions, multiple complementary genetic sequences, or other duplications of portions of the methods discussed herein). Thus, polygenic traits can also be targeted, either by targeting multiple genes or alleles simultaneously, or by targeting a single gene or allele that has a sufficient influence over the polygenic trait to produce the desired effect.

As another possible modification of the systems and methods, some embodiments of the receptor family include WT organisms. Accordingly, the complementary genetic sequence can simply be a WT genetic sequence. Thus, in some cases, only one specialized family of breeding stock is required, and the desired result is obtained by breeding the specialized family with ordinary, WT breeding stock. In some embodiments where this is the case, the specialized family of breeding stock includes a polymorphism in the target gene, such that Cas9 is not directed to that gene locus in the specialized organism, but only to the copy of the target gene in WT organisms. Where this is the case, the polymorphism can include a silent mutation, or it can knock out the copy of that gene (as desired). Stated another way, some embodiments of the donor family are resistant to the effects of Cas9, while in some embodiments, WT organisms are not, thereby enabling WT organisms to be used as the receptor family.

In another possible modification, the guide RNA 30 is included in the complementary sequence construct 26 as opposed to the Cas9 construct 24. Thus, in some such embodiments, the combination of the two constructs (in progeny organisms) is still required to bring the full molecular machinery together.

In accordance with another possible modification, proteins other than Cas9, such as other endonucleases or other proteins configured to perform genetic modifications (particularly modifications that work using a guide RNA or similar mechanism) can be used. For example, some embodiments implement Cas12a, Cas13, or other proteins (which can be included in the Cas9 construct 24) instead of or in addition to Cas9. Thus, the term Cas9 construct is used for convenience and is not necessarily limited to Cas9 (whether inclusive or exclusive of other proteins). Although any suitable protein or other component may be substituted or included in this manner, a non-limiting and illustrative list of examples is as follows: Cas3, Cas8a, Cas5, Cas8b, Cas8c, Cas10d, Cse1, Cse2, Csy1, Csy2, Csy3, GSU0054, Cas10, Csm2, Cmr5, Csx11, Csx10, Csf1, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5) C2c4, C2c8, C2c9, Cas13, Cas13a (C2c2), Cas13b, Cas13c, Cas13d, and Cas13x.1.

In addition to the aforementioned features, some embodiments of the systems and methods discussed include one or more additional features. Indeed, in some embodiments, the process of culling developed organisms that lack a desired trait or that have an undesired trait becomes unnecessary. Culling organisms can be extremely costly, time consuming, arduous, and (in the case of animals, such as male chicks not needed for egg or poultry production) objectionable or inhumane.

Any and all of the components in the Figures, embodiments, implementations, instances, cases, methods, applications, iterations, and other parts of this disclosure can be combined, mixed, or otherwise be used with each other in any suitable manner. Additionally, any component can be removed, separated from other components, modified with or without modification of like components, or otherwise altered together or separately from anything else disclosed herein.

As used herein, the singular forms “a”, “an”, “the” and other singular references include plural referents, and plural references include the singular, unless the context clearly dictates otherwise. For example, reference to a gene includes reference to one or more genes, and reference to organisms includes reference to one or more organisms. In addition, where reference is made to a list of elements (e.g., elements a, b, and c), such reference is intended to include any one of the listed elements by itself, any combination of less than all of the listed elements, or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object-regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope.

What is claimed is: CLAIMS

1. A method of breeding organisms to produce progeny that lack an undesired trait, the method comprising:
obtaining a first parent organism from a donor family of breeding stock;
obtaining a second parent organism from a receptor family of breeding stock; and
mating the first parent organism with the second parent organism to produce a progeny organism,
wherein a combination of genetic material from the first parent organism and the second parent organism in the progeny organism prevents development of the undesired trait due to a non-native genetic construct present in the genetic material.

2. The method of claim 1, wherein at least one of the first parent organism and the second parent organism comprises an allele that corresponds to the undesired trait.

3. The method of claim 2, wherein the combination of genetic material from the first parent organism and the second parent organism in the progeny organism prevents development of the undesired trait by arresting development the progeny organism if the progeny organism comprises the allele that corresponds to the undesired trait.

4. The method of claim 2, wherein, if the progeny organism comprises the allele that corresponds to the undesired trait, the combination of genetic material from the first parent organism and the second parent organism in the progeny organism prevents development of the undesired trait by causing the allele that corresponds to the undesired trait in the progeny organism to be modified.

5. The method of claim 1, wherein the first parent organism comprises the non-native genetic construct, and wherein the non-native genetic construct comprises a Cas9 coding region and a guide RNA coding region.

6. The method of claim 5, wherein the Cas9 coding region is linked to an early embryonic promoter to ensure transcription of a Cas9 protein in an early stage of the progeny organism's development.

7. The method of claim 6, wherein the guide RNA coding region is configured to result in production of guide RNA that is configured to interact with a complementary genetic sequence, and wherein the complementary genetic sequence is not present in DNA of the first parent organism, but is present in DNA of the second parent organism.

8. The method of claim 7, wherein the complementary genetic sequence comprises a polymorphism.

9. The method of claim 8, wherein the polymorphism comprises a silent mutation in a nucleotide sequence of the gene.

10. The method of claim 8, wherein the genetic construct is present in a genetic safe harbor of DNA of the first parent organism.

11. The method of claim 8, wherein the genetic construct is present in a locus of the gene, thereby resulting in a neutralized allele of the gene.

12. The method of claim 5, wherein the construct is located in genetic proximity to a target allele such that activation of the construct is linked to inheritance and activation of the allele.

13. The method of claim 1, wherein the undesired trait comprises a sex of the progeny organism.

14. A method of producing breeding stock configured to produce progeny that lack an undesired trait when a member of a donor family of breeding stock is mated with a member of a receptor family of breeding stock, the method comprising:
obtaining a first target organism;
obtaining a first knock-in construct comprising:
a Cas9 coding region configured to result in production of a Cas9 protein; and
a guide RNA coding region configured to result in production of a guide RNA;
introducing the first knock-in construct into a primordial germ cell of the first target organism using a first CRISPR knock-in procedure;
selectively breeding the first target organism to produce the donor family of breeding stock such that each member of the donor family of breeding stock comprises the first knock-in construct;
obtaining a second target organism;
obtaining a second knock-in construct comprising a nucleotide sequence that is complementary to the guide RNA;
introducing the second knock-in construct into a primordial germ cell of the second target organism using a second CRISPR knock-in procedure; and
selectively breeding the second target organism to produce the receptor family of breeding stock such that each member of the receptor family of breeding stock comprises the second knock-in construct.

15. The method of claim 14, wherein the first knock-in construct further comprises a first homology arm and a second homology arm, wherein each of the first homology arm and the second homology arm are complementary to a genomic safe harbor in a genome of the first target organism.

16. The method of claim 14, wherein the Cas9 coding region of the first knock-in construct is linked to an early embryonic promoter such that the donor family of breeding stock is configured to produce the Cas9 protein during an early stage of embryonic development.

17. The method of claim 14, wherein the second CRISPR knock-in procedure results in a silent mutation in a target gene of the second target organism, wherein the silent mutation comprises an insertion of the second knock-in construct.

18. A bifurcated breeding stock for producing progeny that lack an undesired trait, the bifurcated breeding stock comprising:
a donor family comprising a first construct that comprises:
a Cas9 coding region configured to result in production of a Cas9 protein; and
a guide RNA coding region configured to result in production of a guide RNA configured to associate with the Cas9 protein; and
a receptor family comprising a second construct that comprises:
a nucleotide sequence that is complementary to the guide RNA,
wherein breeding members of the donor family with other members of the donor family produces donor family offspring, and breeding members of the receptor family with other members of the receptor family produces receptor family offspring, but wherein breeding a member of the donor family with a member of the receptor family produces only progeny that lack the undesired trait due to an interaction between the Cas9 protein and the nucleotide sequence that is complementary to the guide RNA in progeny that would otherwise have the undesired trait.

19. The bifurcated breeding stock of claim 18, wherein the interaction between the Cas9 protein and the nucleotide sequence that is complementary to the guide RNA in progeny that would otherwise have the undesired trait results in arrested embryonic development of the progeny that would otherwise have the undesired trait, and wherein progeny that would not otherwise have the undesired trait are not subjected to the interaction between the Cas9 protein and the nucleotide sequence that is complementary to the guide RNA.

20. The bifurcated breeding stock of claim 18, wherein the undesired trait is a sex of the progeny, and wherein at least one of the first construct and the second construct is present within a genetic sequence of a sex chromosome.
